(19) United States
(12) Patent Application Publication
(10) Pub. No.: US 2023/0178173 A1
(43) Pub. Date: Jun. 8, 2023

(54) SYSTEMS AND METHODS FOR GUT MICROBIOME PRECISION MEDICINE

(71) Applicant: The Regents of the University of California, Oakland, CA (US)

(72) Inventors: Mohammad R. Kaazempur-Mofrad, Lafayette, CA (US); Mohammad Soheilypour, Danville, CA (US)

(21) Appl. No.: 17/995,250

(22) PCT Filed: Mar. 30, 2021
(86) PCT No.: PCT/US21/24963

§ 371 Date: Sep. 30, 2022

Related U.S. Application Data
(60) Provisional application No. 63/115,870, filed on Nov. 19, 2020.
(60) Provisional application No. 63/001,795, filed on Mar. 30, 2020.

(51) Int. Cl. G16B 15/30; G16B 40/20; G16B 5/00; G06N 3/04; G16C 20/30

(52) U.S. Cl. CPC G16B 15/30; G16B 40/20; G16B 5/00; G06N 3/04; G16C 20/30

(57) ABSTRACT

Methods and systems are provided for simulating metabolic interactions between a microbiome and a chemical compound. In one embodiment, a method includes predicting, with a trained deep neural network, a plurality of enzymes potentially responsible for metabolism of a chemical compound, generating a three-dimensional individual-specific model of a microbiome including one or more microorganisms associated with the plurality of enzymes, and simulating, with the three-dimensional individual-specific model, metabolism of the chemical compound in the microbiome over time. In this way, the individual-specific metabolism of chemical compounds, such as drug compounds, in microbiomes, such as human gut microbiomes, may be reliably predicted in a high-throughput fashion while accounting for three-dimensional compound structure.

### CROSS REFERENCE TO RELATED APPLICATIONS

The present application claims priority to U.S. Provisional Application No. 63/115,870, entitled “SYSTEMS AND METHODS FOR GUT MICROBIOME PRECISION MEDICINE”, and filed on Nov. 19, 2020. This present application also claims priority to U.S. Provisional Application No. 63/001,795, entitled “GUT MICROBIOME MODEL”, and filed on Mar. 30, 2020. The entire contents of the above-listed applications are hereby incorporated by reference for all purposes.

### GOVERNMENT SUPPORT

This invention was made with government support under Grant Numbers ES026541 and GM130228 awarded by the National Institutes of Health. The government has certain rights in the invention.

### FIELD

The present description relates generally to a computational platform for evaluating interactions between a drug compound and a gut microbiome.

### BACKGROUND

Since orally-administered drugs spend a considerable amount of time in the vicinity of the complex and dynamic community of microorganisms residing in the intestines, called the gut microbiome, they could potentially interact with these microorganisms. As a result of interacting with the gut microbiome, many pharmaceuticals are transformed into metabolites with altered disposition, efficacy, and toxicity. These drugs range from common drugs such as acetaminophen to life-saving drugs such as colorectal cancer chemotherapeutic and prodrug irinotecan.

Additionally, the highly-variable functional and compositional landscape of gut microbial communities across individuals further contributes to patient-to-patient variations in drug response (e.g., efficacy and toxicity), which could result in a need for alternate dosing or medication strategies. As a result, understanding the interplay between the gut microbial ecosystem and therapeutics is attracting increasing attention in the pharmaceutical industry.

### SUMMARY

In one embodiment, a method comprises predicting, with a trained deep neural network, a plurality of enzymes potentially responsible for metabolism of a chemical compound, generating a three-dimensional individual-specific model of a microbiome including one or more microorganisms associated with the plurality of enzymes, and simulating, with the three-dimensional individual-specific model, metabolism of the chemical compound in the microbiome over time. In this way, the individual-specific metabolism of chemical compounds, such as drug compounds, by microbiomes, such as human gut microbiomes, may be reliably predicted in a high-throughput fashion while accounting for three-dimensional compound structure.

It should be understood that the brief description above is provided to introduce in a simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows a block diagram illustrating an example computing system providing a personalized computational platform for predicting individual-specific response of the gut microbiome to various drugs, according to an embodiment;

FIG. 2 shows a block diagram illustrating an example module architecture for a personalized computational platform for predicting individual-specific response of the gut microbiome to various drugs, according to an embodiment;

FIG. 3 shows a block diagram illustrating an example method for predicting potential enzyme(s) and microorganism(s) responsible for metabolism of a target drug, according to an embodiment;

FIG. 4 shows a high-level flow chart illustrating an example method for predicting gut microbiome-mediated drug metabolism, according to an embodiment;

FIG. 5 shows a high-level flow chart illustrating an example method for identifying potential enzymes that metabolize a target compound, according to an embodiment;

FIG. 6 shows a high-level flow chart illustrating an example method for processing omics data to identify microbial species and reconstruct metabolic models, according to an embodiment;

FIG. 7 shows a set of graphs illustrating that gut microbiome simulations with the personalized computational platform are stable over twenty-four hours according to different metrics including Shannon diversity index and Aitchison distance;

FIG. 8 shows a set of graphs illustrating example measurements from in silico experiments versus in vitro experiments, according to an embodiment; and

FIG. 9 shows a set of graphs illustrating that the personalized computational platform accurately predicts microbial metabolism of diltiazem by three fecal samples from three different healthy individuals over twenty-four hours, according to an embodiment.

### DETAILED DESCRIPTION

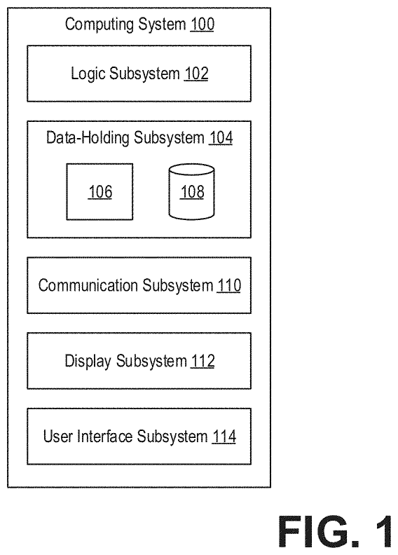

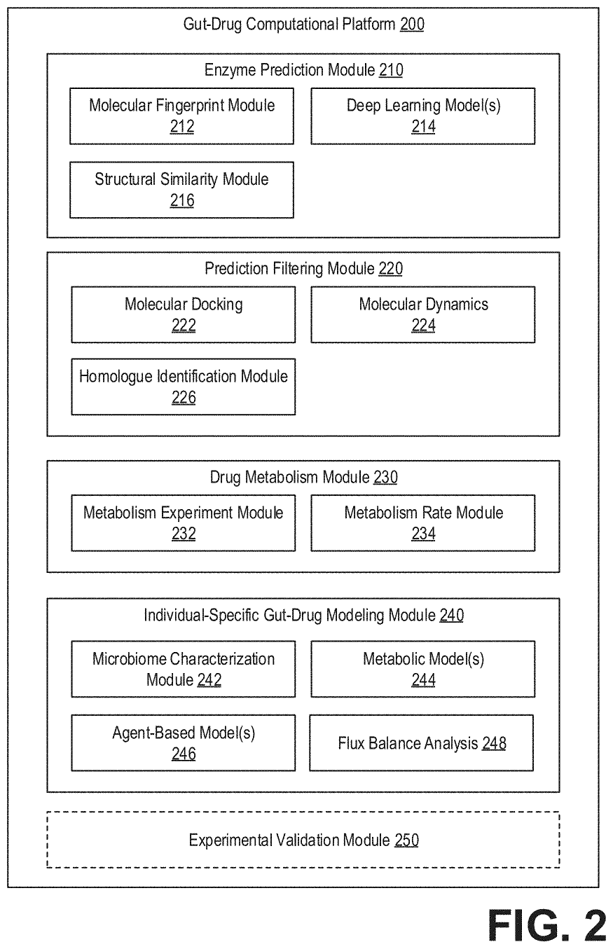

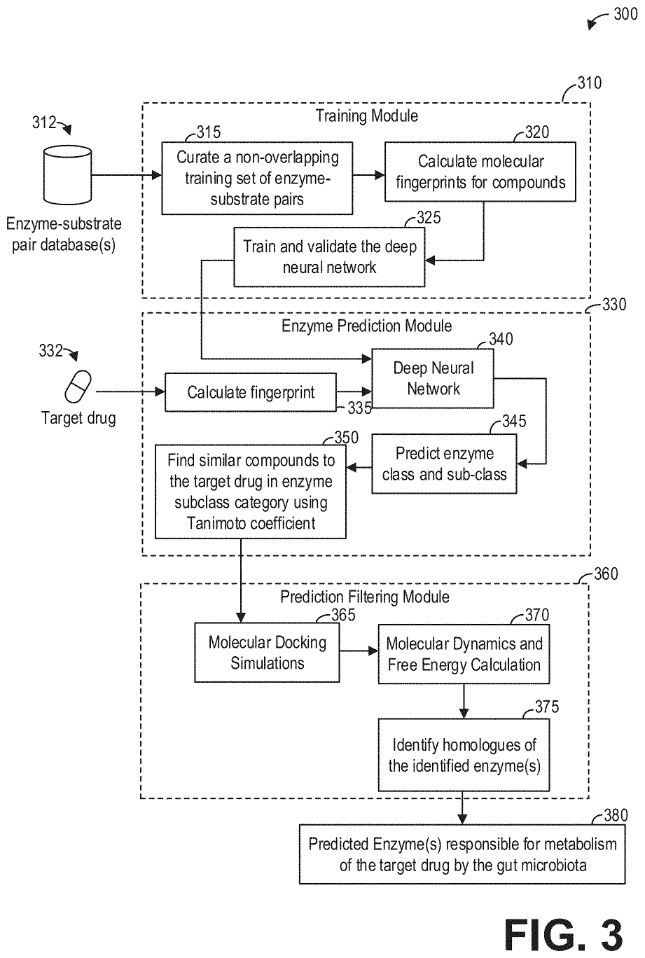

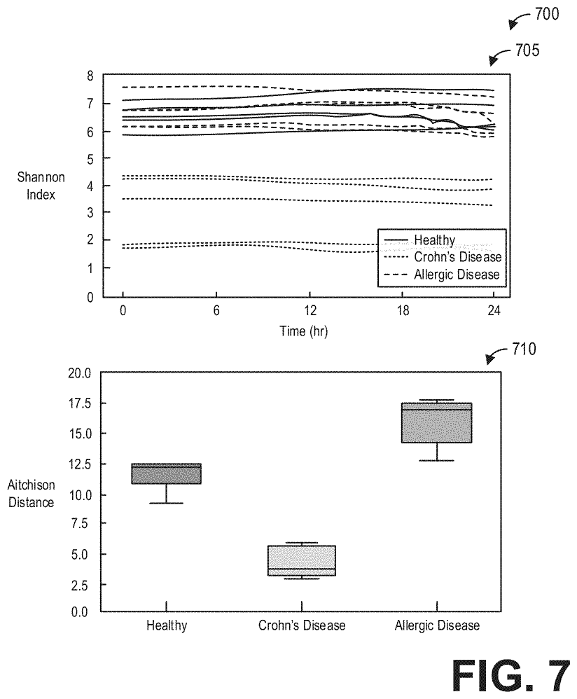

The following description relates to a computational platform for predicting gut microbiome-mediated drug metabolism. A computing system, such as the computing system shown in FIG. 1, may provide a computational platform, such as the computational platform shown in FIG. 2, configure to perform high-throughput testing of drugs against potential drug-metabolizing bacteria. In particular, the platform enables accurate testing of hundreds of chemical compounds, including but not limited to drug compounds, against thousands of potential compound-metabolizing bacteria. The platform further enables predictions of drug metabolism that integrates human metabolic processes as well as parallel microbial metabolism. Further still, the platform incorporates inter-individual variability to explore the mechanistic link between microbial genomic content present in the gut and the associated drug metabolizing capacity. The methods for the computational platform, as shown in FIGS. 3-5, integrate three-dimensional modeling methods and high-throughput fingerprint-based techniques to achieve accurate predictions of drug-metabolizing enzymes within the gut microbiome. Additionally, these methods further predict individual-specific effect of gut microbiome on drug metabolism by accounting for metagenomics, metatranscriptomic, and/or metaproteomic data. The systems and methods provided herein thus capture the individual-specific composition and functional landscape of the human gut microbiome, enable the exploration of the interplay between drugs and the gut microbiota, and are experimentally validated on multiple levels for reliable predictions. Comparisons of the simulated or in silico experiments of drug metabolism against more traditional in vitro experiments, as shown in FIGS. 7-9, demonstrate that the systems and methods provided herein achieve highly accurate predictions, thereby resulting in a more detailed understanding of drug metabolism.

Turning now to the drawings, FIG. 1 shows a block diagram illustrating an example computing system 100 providing a personalized computational platform for predicting individual-specific effect of the gut microbiome on various drugs. It should be appreciated that the architecture of the computing system 100 is exemplary and non-limiting, and that other computer architectures may be used for a computing device without departing from the scope of the present disclosure. In different embodiments, the computing system 100 may comprise a mainframe computer, a server computer, a desktop computer, a laptop computer, a tablet computer, a network computing device, a mobile computing device, a mobile communication device, and so on. As depicted, the computing system 100 comprises a logic subsystem 102 and a data-holding subsystem 104. The computing system 100 may further include a communication subsystem 110, a display subsystem 112, and a user interface subsystem 114.

The logic subsystem 102 may include one or more physical devices configured to execute one or more instructions. For example, the logic subsystem 102 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem 102 may include one or more processors that are configured to execute software instructions. In some examples, the logic subsystem 102 may include one or more hardware and/or firmware logic machines configured to execute hardware and/or firmware instructions. Processors of the logic subsystem 102 may be single core or multi-core, and the programs executed thereon may be configured for parallel or distributed processing. The logic subsystem 102 may optionally include individual components that are distributed throughout two or more devices, which may be remotely located and/or configured for coordinated processing. One or more aspects of the logic subsystem 102 may be virtualized and executed by remotely-accessible networked computing devices configured in a cloud computing configuration.

The data-holding subsystem 104 may include one or more physical, non-transitory devices configured to hold data and/or instructions executable by the logic subsystem 102 to implement the herein-described methods and processes. When such methods and processes are implemented, the state of data-holding subsystem may be transformed (for example, to hold different data).

The data-holding subsystem 104 may include removable media and/or built-in devices. Data-holding subsystem 104 may include optical memory (for example, CD, DVD, HD-DVD, Blu-Ray Disc, and so on), and/or magnetic memory devices (for example, hard disk drive, floppy disk drive, tape drive, MRAM, and so on), and the like. The data-holding subsystem 104 may include devices with one or more of the following characteristics: volatile, nonvolatile, dynamic, static, read/write, read-only, random access, sequential access, location addressable, file addressable, and content addressable. In some embodiments, the logic subsystem 102 and the data-holding subsystem 104 may be integrated into one or more common devices, such as an application specific integrated circuit or a system on a chip. In other embodiments, the data-holding subsystem 104 may include individual components that are distributed throughout two or more devices, which may be remotely located and accessible through a networked configuration.

When included, the communication subsystem 110 may be configured to communicatively couple the computing system 100 with one or more other computing devices. The communication subsystem 110 may include wired and/or wireless communication devices compatible with one or more different communication protocols. As non-limiting examples, the communication subsystem 110 may be configured for communication via a wireless telephone network, a wireless local area network, a wired local area network, a wireless wide area network, a wired wide area network, and so on. In some examples, the communications subsystem 110 may enable the computing system 100 to send and/or receive messages to and/or from other computing systems via a network such as the public Internet.

When included, the display subsystem 112 may be used to present a visual representation of data held by data-holding subsystem 104. As the herein-described methods and processes change the data held by the data-holding subsystem 104, and thus transform the state of the data-holding subsystem 104, the state of display subsystem 112 may likewise be transformed to visually represent changes in the underlying data. The display subsystem 112 may include one or more display devices utilizing any type of display technology. Such display devices may be combined with the logic subsystem 102 and/or the data-holding subsystem 104 in a shared enclosure, or such display devices may comprise peripheral display devices.

When included, the user interface subsystem 114 may include one or more physical devices configured to facilitate interactions between a user and the computing system 100. For example, the user interface subsystem 114 may comprise one or more user input devices including but not limited to a keyboard, a mouse, a camera, a microphone, a touch screen, and so on.

As described further herein, the computing system 100 provides a personalized computational platform for predicting individual-specific effect of the gut microbiome on various drugs. To that end, the data-holding subsystem 104 may store a computational platform 106 for predicting individual-specific effect of the gut microbiome on various drugs. An example computational platform 106 is described further herein with regard to FIG. 2. The data-holding subsystem 104 may further store one or more databases 108, including one or more of a database of gut proteins such as the Unified Human Gastrointestinal Protein (UHGP) database, a database of substrates for gut microbiome-associated enzymes, a database of protein sequence and functional information such as the UniProt database, a database for genes and genomes such as the Kyoto Encyclopedia of Genes and Genomes (KEGG) database, a database for enzyme structural and functional information such as the BRENDA database, and so on.

FIG. 2 shows a block diagram illustrating an example module architecture for a personalized computational platform 200 for predicting individual-specific effect of the gut microbiome on various drugs, according to an embodiment. The personalized computational platform 200 may be implemented as the computational platform 106 in the computing system 100, as an illustrative and non-limiting example. It should be appreciated that the modules of the computational platform 200 are exemplary and non-limiting, and that the computational platform 200 may be implemented with other modules and sub-modules without departing from the scope of the present disclosure.

The computational platform 200 comprises a plurality of modules, including an enzyme prediction module 210 configured to predict one or more enzymes that may metabolize a target drug compound, a prediction filtering module 220 configured to identify best enzyme candidates from the predicted enzymes output by the enzyme prediction module 210, a drug metabolism module 230 to identify the metabolism rate of the target drug by target microorganism(s), an individual-specific gut-drug modeling module 240 configured to simulate metabolism of the target drug compound in an in silico gut microbiome, and optionally an experimental validation module 250 configured to validate modules of the computing platform 200 based on experimental data.

The enzyme prediction module 210 may comprise a molecular fingerprint module 212 configured to calculate molecular fingerprints comprising representations of compounds. The molecular fingerprints representing molecular structures may be translated to machine-readable features for input to one or more deep learning models. As illustrative and non-limiting examples, different molecular fingerprint approaches, including PubChem 2D fingerprints and molecular access system (MACCS) keys, may be used for the molecular fingerprints. PubChem 2D is an 881 dimension binary vector in which each bit represents a specific element, functional group, ring system or other discrete chemical entity. MACCS keys are 166 bit structural key descriptors in which each bit is associated with a SMARTS pattern, where SMARTS is a language that allows specifying substructures by providing a number of primitive symbols describing atomic and bond properties. The molecular fingerprint module 212 may use PaDEL to calculate fingerprints. Each molecular fingerprint can be evaluated separately during the training process to identify the best structural descriptor.

The enzyme prediction module 210 may further comprise a deep learning model module 214 comprising one or more deep learning models configured to predict potential enzyme(s) and their associated microorganism(s) that may metabolize a target therapeutic drug. The one or more deep learning models of the deep learning model module 214 may comprise one or more convolutional neural networks (CNNs), as an illustrative and non-limiting example. As an example, the CNN structure may be designed in Python using Tensorflow and Keras packages. The structure of the CNN may be determined by examining four main hyperparameters, namely window size of filters, number of filters for each window size, number of hidden layers, and number of nodes in each hidden layer of the fully-connected layer. Hyperparameter values that generate the highest macro precision will be used to model the CNN. In the embedding layer, an embedding matrix with a dimension of N×2 (0 or 1), where N is the length of fingerprint (e.g. 881 for PubChem 2D), will be generated from an input molecular fingerprint using one-hot encoding method. The CNN may use rectified linear units (ReLU) for activation functions, and dropout layers are placed directly after any fully connected layers to prevent overfitting. Dropout layers are at uniform intervals between 0.1 and 0.5. An Adam optimizer can be used to speed up convergence of the models. The performance of the CNN may be evaluated using a 70-10-20 split of the training data. Macro precision and macro recall performance metrics can be used to validate and test the CNN.

The input to the deep learning model of the deep learning model module 214 may comprise a molecular fingerprint of the target compound and the output may comprise an identified enzyme classification such as an Enzyme Commission (EC) number, such as a class or subclass of the predicted enzyme. As a particular, example, the deep learning model may output a two-digit EC class and subclass number to which the predicted enzyme(s) belong.

The enzyme prediction module 210 may further identify candidate enzyme(s) from the identified enzyme classification output by the deep learning model module 214 using the structural similarity module 216. For example, the enzyme prediction module 210 may identify a class and a sub-class of candidate enzyme(s) by using the deep learning model module 214 for a given target drug. Compound structural similarity may serve as a proxy for enzyme sharing. Therefore, the structural similarity module 216 may identify the sub-subclass and serial number of candidate enzyme(s) by running a molecular similarity search against substrates associated with the predicted subclass. The similarity between two substrates may be defined by the Tanimoto coefficient of two-dimensional (2D) fingerprints of the two compounds. The Tanimoto coefficient TC between compounds A and B may be calculated according to:

$$T C = \frac{Z}{x + y - z} ,$$

where x is the number of bits set to 1 in compound A, y is the number of bits set to 1 in compound B, and z is the number of bits set to 1 in both compounds A and B. The enzyme prediction module 210 may perform a similarity search using OpenBabel, as an illustrative and non-limiting example. Thus, through the association between predicted enzymes and their corresponding microorganisms, microorganisms that potentially metabolize a target drug compound may be identified.

As mentioned hereinabove, the prediction filtering module 220 provides a three-dimensional (3D) molecular structure modeling pipeline configured to identify best enzyme candidates from the predicted enzymes output by the enzyme prediction module 210 by filtering the predicted drug-metabolizing enzyme(s). In some examples, the prediction filter module 220 comprises a molecular docking module 222 and a molecular dynamics module 224. The molecular docking module 222 may be configured to identify binding sites and interaction orientations through molecular docking. For example, the molecular docking module 222 may obtain crystal structure(s) of the predicted enzyme(s) from an enzyme database, such as the BRENDA enzyme database which includes tens of thousands of three-dimensional enzyme structures across thousands of EC classes. The molecular docking module 222 may include the Glide docking program to perform accurate docking analysis of enzymes and substrates. The target drug compound may be docked considering its flexibility by ligand conformational sampling method implemented in Glide, for example. The molecular docking module 222 may obtain multiple poses per enzyme-drug pair. The five best poses for a given enzyme-drug pair may be determined by a highest value of GlideScore, the empirical scoring function in Glide that approximates the ligand binding free energy.

The molecular dynamics module 224 performs molecular dynamics simulations to identify the best enzyme candidates. Performing molecular dynamics simulations may identify false positive predictions of enzymes that may not have been filtered by the enzyme prediction module 210, and provide mechanistic insights about enzyme-drug binding by identifying key contacts between substrates and the enzyme catalytic pocket. The molecular dynamics module 224 may use GROMACS and CHARMM36 force field for molecular dynamics simulations, as illustrative and non-limiting examples. A regular molecular dynamics protocol may include solvation, neutralization of the net charge, minimization, and equilibration for one nanosecond using the NPT ensemble. The molecular dynamics module 224 may use a particle mesh Ewald method to calculate electrostatic interactions. The molecular dynamics module 224 may apply periodic boundary conditions in all three directions, use a time step of 2 femtoseconds, and maintain temperature at 310K and pressure at 1 bar. All molecular dynamics simulations may be run for one microsecond, for example, using the NPT ensemble. The molecular dynamics module 224 may use a visual molecular dynamics package for post-simulation visualization and analysis.

The molecular dynamics module 224 may output several readouts including root mean square deviation (RMSD), binding energy, and free energy, which may be calculated from molecular dynamics simulation trajectories using umbrella sampling. Free energy is one of the main governing factors in drug discovery and drug design. The umbrella sampling method is one of the most accurate methods for free energy calculation. The initial configurations can be prepared for sampling each geometrically possible binding orientation. At least twenty histograms can be generated for each binding event. In some examples, different umbrella potentials starting from 1000 kJ mol⁻¹ nm⁻² can be used until smooth and localized histograms are generated. All histograms can be combined using the Weighted Histogram Analysis Method (WHAM) algorithm. The reaction coordinate of the umbrella sampling can be defined as the center of mass distance between the Ca atoms of the binding residues of enzyme (the pulling group) and binding residues of substrate (the reference group). The reference step for umbrellas can be 0.4 A° to optimize the overlap of the above-mentioned histograms. At each umbrella, a one nanosecond sampling can be performed. The final Potential Mean Force and histograms can be calculated using Grossfield's WHAM code. A free energy profile can be obtained for each binding orientation, and the profile with the largest free energy drop corresponds to the best binding orientation. This information then can be used to measure association and dissociation constants, which are used for computing binding affinities.

The prediction filtering module 220 may further comprise a homologue identification module 226 comprising one or more computational methods to identify homologues of the enzyme(s). As an example, the homologues of the enzyme(s) could be identified using MetaPhOrs, a public repository of phylogeny-based orthologs and paralogs that were computed using phylogenetic trees available in twelve public repositories. Sequence-based homologue search can be performed using the cut-off values of Identity>80% and E-value<10⁻¹⁵ to identify, microorganisms that encode homologues of the target enzyme. These cut-off values may be further calibrated.

The drug metabolism module 230 comprises a metabolism experiment module 232 and a metabolism rate module 234 to identify the metabolism rate of the target drug by target microorganism(s). The metabolism experiment module 232 may characterize the metabolism rate of the target drug using monoculture experiments. For example, experiments may be conducted in serum bottles under anaerobic conditions. Microorganisms identified by prediction filtering module 220 to metabolize the target drug may be grown in Bacto Brain Heart Infusion broth or modified Gifu Anaerobic Medium (mGAM) broth or Gut Microbiota Medium (GMM). The bacteria may be grown in a 125 mL sterile serum bottle with 50 mL of media inside an anaerobic chamber under a headspace of 5% H2, 20% CO2 and balance N2. After forty-eight hours of growth, bacterial cells may be used to perform degradation studies with different concentrations of the respective drug. Samples may be collected every 4 hours until 48 hours and may be analyzed using HPLC-MS for drugs and metabolites.

The metabolism rate module 234 obtains the data for drug concentration over time from experiment module 232. Rate of drug concentration change between each two data points may be identified by dividing the concentration change by the time between the two data points. Rates of drug concentration change will be then plotted for each concentration of the drug. Concentration-dependent drug metabolism rate may then be identified by fitting a linear curve to this plot (i.e., rate of drug-concentration change versus drug concentration). The slope of the fitted curve would then be used as the average rate for drug metabolism.

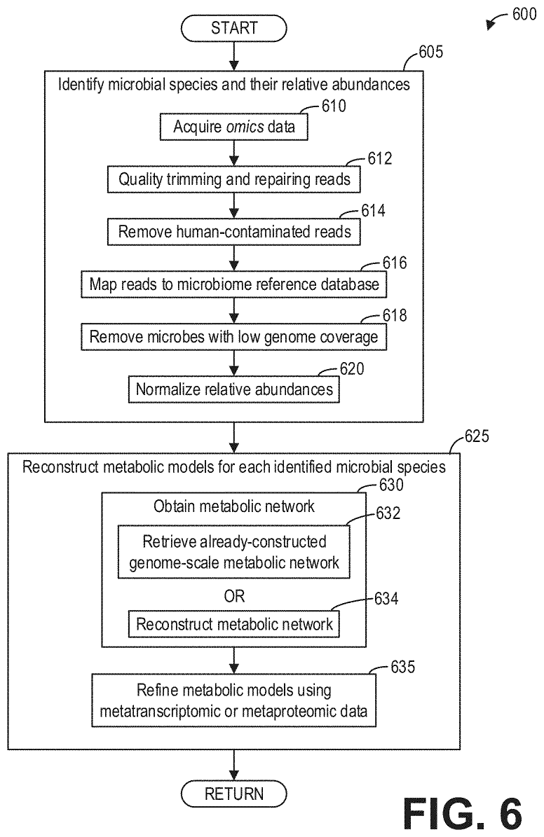

The individual-specific gut-drug modeling module 240 comprises a microbiome characterization module 242, a metabolic model module 244, an agent-based model module 246, and a flux balance analysis module 248. The microbiome characterization module 242 may extract types and abundance of microbial species from omics data. For example, FIG. 6 shows an example method 600 for the microbiome characterization module 242. Method 600 begins at 605, where method 600 identifies microbial species and their relative abundances. To that end, method 600 obtains raw metagenomic data either through sequencing the target microbiome or via the NCBI sequence read archive (SRA). At 612, method 600 quality trims the reads using Trimmomatic and then re-pairs the reads using the BBmap repair tool. At 614, method 600 removes human contaminant sequences by mapping the paired reads to human reference genome build 38 (GRCh38) using Burrows-Wheeler Aligner (BWA). Cross-mapped reads (reads mapped to multiple positions) may be filtered out by discarding mapped reads with a low-quality score using SAMtools. At 616, method 600 then maps the pre-processed reads to a reference gut microbiome database. At 618, method removes microbes with low genome coverage. For example, the abundance of each microbial species may be calculated by adding up the sequence length of reads mapped to a unique region of a species' genome, normalized by the total size of the species' genome. A minimum genome coverage (for example, 1%) may be assigned for each identified microorganism to reduce the number of false positives. At 620, the resulting coverages for each microorganism may be normalized to 1 Gb to obtain relative microbe abundances.

After identifying the microbial species and their relative abundances at 605, method 600 proceeds to 625, where method 600 reconstructs metabolic models for each identified microbial species. Genome-scale metabolic models relate metabolic genes with metabolic pathways. Thus, at 630, the metabolic model module 244 retrieves or reconstructs the metabolic models associated with the microorganisms identified by microbiome characterization module 242. The metabolic model module 242 may use metabolic model datasets at 632, in some examples, or in other examples the metabolic model module 242 may, at 634, use metabolic network reconstruction methods or tools, such as the CarveMe tool to build metabolic models using reference genomes. After obtaining the metabolic networks, method 600 continues to 635. At 635, method 600 further refines the metabolic models using metatranscriptomic or metaproteomic data. First, gene or protein expression data is binarized into on and off states. Subsequently, these states are used to modify metabolic pathways by mapping to corresponding genome-scale metabolic network reconstructions. The reconstructed metabolic models may be associated with a corresponding agent type in the agent-based model(s) module 246. Method 600 then returns.

Referring again to FIG. 2, the agent-based model(s) module 246 constructs an individual-specific model of the target gut microbiome in interaction with the target drug compound. The primary inputs to the model may be microbial species identified in microbiome characterization module 242, relative abundance of each microorganism identified in microbiome characterization module 242, metabolic networks associated with each microorganism identified in metabolic model(s) module 244, and metabolites that should be present to support these metabolic pathways. Additional inputs to the model may include simulation parameters such as the size of the system (e.g., in micrometers), the time step (e.g., in seconds), and the number of desired simulation steps as well as molecular fields in the system (e.g., the target drug compound), their diffusion coefficients, and their initial concentrations. The agent-based model(s) module 246 may then construct the three-dimensional environment of the simulation where agents (representing microorganisms) are distributed randomly, with each microbe given random initial biomass according to a median cell dry weight (e.g. 0.489 pg) and a dry weight deviation (e.g. 0.132 pg).

The modeling environment in agent-based model(s) module 246 may be discretized at the molecular scale and the initial concentration of molecular fields may be assigned to each grid cell. Molecular species (e.g., metabolites and drugs) may be modeled using ordinary differential equations (ODEs) and allowed to diffuse between boxes with the diffusion of molecules governed by Fick's Second Law:

$$\frac{\partial [ C ]}{\partial T} = D ( \frac{{\partial }^{2} [ C ]}{\partial {x}^{2}} + \frac{{\partial }^{2} [ C ]}{\partial {y}^{2}} + \frac{{\partial }^{2} [ C ]}{\partial {z}^{2}} ) .$$

Diffusion may be modeled using the algorithm proposed by Grajdeanu. Based on this algorithm the concentration in each grid cell depends on the concentration in neighboring grid cells, the distance between cells, and the diffusion coefficient, which may be calculated according to:

$${d}_{j} = \vert {x}_{i} - {x}_{i}^{j} \vert ,$$

$$C ( {x}_{i} ) = C ( {x}_{i} ) + A {\sum }_{j = 1}^{n} ( C ( {x}_{i}^{j} ) - C ( {x}_{i} ) ) {e}^{- \frac{{d}_{j}^{2}}{D}} , and$$

$$A {\sum }_{j = 1}^{n} {e}^{- {d}_{j}^{2} / D} = 1 .$$

The movement of agents (representing microbes) may be modeled by random walk (suggested for time steps greater than 30 minutes) or biophysical flagellar movement, such as running and tumbling. A pairwise collision force may be applied to all overlapping microorganisms to avoid collision of diffusing bacterial agents. The magnitude of this force is proportional to the log of the ratio of the distance between two bacteria centers and the sum of their radii.

The agent-based model(s) module 246 then runs the simulation, also referred to herein as the in silico experiment, for the desired number of time steps. At each time step, a range of data may be stored such as coordinates of microorganisms, cell population, and the concentration of molecular fields. Microorganisms may be represented by autonomous agents possessing cellular characteristics including growth, division, and migration. Microorganism growth, death, and division rules and rates may be naturally calculated from metabolic interactions or implemented based on experimental studies of morphogenesis in individual bacteria. In addition to characteristics of agents, agent-based model tools may provide other aspects of the simulation such as environmental boundaries, physical factors (e.g., crowding and steric repulsion), and collision detection.

The flux balance analysis module 248 uses flux balance analysis to predict metabolic interactions of microorganisms with the environment, and hence, identify their microbial growth. The flux balance analysis module 248 calculates the flow of metabolites through biochemical reactions in a metabolic network. The fluxes may be computed by optimizing an objective function,

Z=c^{T}v,

where ν is the vector of target fluxes. The linear programming problem is therefore to solve

S·v=0,

where S is an m×n stoichiometric matrix of biochemical reactions with m compounds and n reactions, subject to lower and upper bounds for the vector ν and a linear combination of fluxes Z as the objective function. Each agent may be assigned its metabolic models according to agent type. A linear programming (LP) solver such as GLPK (GNU Linear Programming Kit) or COIN-OR Linear Programming (CLP) may be used to solve LP problems for FBA. Lower bounds of fluxes may be updated according to the local concentration of metabolites in the vicinity of the microorganism. At each time step, LP solver may solves LP problems for each microorganism and updates environmental concentrations of the metabolites that are involved in exchange metabolic interactions. Additionally, the biomass accumulated by an individual agent may be updated according to an exponential growth model using the optimal biomass flux computed by FBA:

Biomassₜ₊ᵢ=Biomassₜ+v_{biomass}×Biomassₜ×dt.

Once accumulated biomass reaches a maximal dry weight (e.g. 1.172 pg), microbes replicate. When the accumulated biomass drops below a minimal dry weight (e.g. 0.083 pg), microorganisms die.

Drug metabolism may be governed by ODEs using drug metabolism rate identified in drug metabolism module 230. At each time step, molecular fields may be evaluated and field concentrations may be updated according to metabolism of target drug by microorganisms identified in prediction filtering module 220.

The experimental validation module 250 is configured to use experimental data to validate the computational platform 200. For example, the experimental validation module 250 may validate predicted metabolism and drug concentration changes over time. In one example, the experimental validation module 240 may use experimental data obtained via ex vivo metabolism of target drug(s) by fecal samples including microorganisms predicted by the enzyme prediction module 210 and filtered by the prediction filtering module 220. After pre-processing, a glycerol stock of the fecal sample may be used to inoculate in modified Gifu Anaerobic Medium (mGAM) broth, or modified Gifu Anaerobic Medium (mGAM) broth or Gut Microbiota Medium (GMM), in order to grow for 48 hours. After the growth period, an aliquot of the culture may be used for degradation kinetics of the target drug(s) in the media. Cultures may be incubated for 24 hours in an anaerobic chamber. Experiments may be performed in triplicate to obtain statistically significant and reproducible results. Samples may then be collected and centrifuged to remove any fecal bacteria, at multiple data points such as 0, 1 h, 2 h, 6 h, 8 h, 12 h, 16 h and 24 hours and may then be analyzed using HPLC-MS.

FIG. 3 shows a block diagram illustrating an example method 300 for predicting potential enzyme(s) and microorganism(s) responsible for metabolism of a target drug, according to an embodiment. In particular, the method 300 relates to training a deep neural network to predict potential enzymes and their associated microorganisms that may metabolize a target therapeutic drug, and deploying the trained deep neural network to generate predictions for a target drug.

A training module 310 is configured to train a deep neural network of the deep learning model module 214, for example. In order to develop a reliable deep learning model for gut-mediated drug metabolism, the training module 310 assembles a training dataset of compounds that are metabolized by gut microbiome enzymes. As an example, the training module 310 imports enzyme-substrate pairs from one or more enzyme-substrate pair database(s) 312. The enzyme-substrate pair database(s) 312 may include, as an illustrative and non-limiting example, the Unified Human Gastrointestinal Protein (UHGP) catalog, a database of gut proteins containing over 171 million proteins encoded by gut microbiota. The database(s) 312 may further include a database of protein sequence and functional information, such as the UniProt database. At 315, the training module 310 curates a non-overlapping training set of enzyme-substrate pairs from the data imported from the enzyme-substrate pair database(s) 312. For example, the training module 310 may use the UniProt database to identify metabolic enzymes from the UHGP database. In particular, the reviewed enzymatic protein sequences with their corresponding Enzyme Commission (EC) numbers may be obtained from the UniProt database. The training module 310 may use this dataset as a reference to carry out protein BLAST (BLASTp) alignment of all proteins from the UHGP database. Based on the results of the BLASTp alignment query which comprise regions of similarity between biological sequences, the training module 310 identifies or selects at least one gut microbiome protein as a top result based on cut-off values for the results. As an illustrative example, the training module 310 may select a gut microbiome protein from the results with values of Identity above 40%, Query coverage above 80%, and Expect value (E-value) below 10⁻¹⁵. The protein sequences that match a hit for a gut microbiome protein may be assigned with the EC number of the corresponding best hit. Further, to link the identified enzymes to the set of compounds they metabolize, the training module 310 may use a dataset of compound identifiers with links to EC numbers. For example, the training module 310 may import data from the Kyoto Encyclopedia of Genes and Genomes (KEGG) database, for example via KEGGREST, including KEGG compound identifiers with links to EC numbers. In order to include only relevant molecules in biochemical transformations, the training module 310 may remove cofactors and other supporting molecules for enzyme functioning such as water, ions, ATP, and so on. The resulting set of unique compounds linked to metabolizing enzymes that are tagged with their EC numbers and associated microorganisms thus forms the training dataset.

At 320, the training module 310 calculates molecular fingerprints for compounds. In order to use the training dataset curated at 315, the training module 310 may translate molecular information about substrates into machine-readable features. To that end, molecular fingerprints may be used to represent molecular structures. The training module 310 may use molecular fingerprints such as PubChem 2D fingerprints, wherein each PubChem 2D fingerprint comprises an 881-dimension binary vector in which each bit represents a specific element, functional group, ring system, or other discrete chemical entity. The training module 310 may additionally or alternatively use molecular access system (MACCS) keys for molecular fingerprints, wherein each MACCS key comprises a 166-bit structural key descriptor in which each bit is associated with a SMARTS pattern. The training module 310 may use PaDEL to calculate molecular fingerprints. Each molecular fingerprint may be separately evaluated during the training process to identify the best structural descriptor.

After curating the training set at 315 and calculating molecular fingerprints for compounds at 320, the training module 310 trains and validates the deep neural network at 325. As an illustrative and non-limiting example, the deep neural network may comprise a CNN, and the CNN structure may be designed in Python using Tensorflow and Keras packages. The structure of the deep neural network may be determined by examining four main hyperparameters, namely window size of filters, number of filters for each window size, number of hidden layers, and number of nodes in each hidden layer of the fully-connected layer. Hyperparameter values that generate the highest macro precision will be used to model the deep neural network. In the embedding layer, an embedding matrix with a dimension of N×2 (0 or 1), where N is the length of the molecular fingerprint (e.g. 881 for PubChem 2D), will be generated from an input molecular fingerprint using one-hot encoding method. The deep neural network may use rectified linear units (ReLU) for activation functions, and dropout layers are placed directly after any fully connected layers to prevent overfitting. Dropout layers are at uniform intervals between 0.1 and 0.5. An Adam optimizer can be used to speed up convergence of the models. The performance of the deep neural network may be evaluated using a 70-10-20 split of the training dataset. Macro precision and macro recall performance metrics can be used to validate and test the deep neural network.

The enzyme prediction module 330 calculates a molecular fingerprint at 335 for a target drug 332 to be simulated. The enzyme prediction module 330 may use the trained deep neural network 340 obtained from 325 for the molecular fingerprint obtained at 335 for the target drug 332, in order to obtain a prediction of a class and a sub-class of candidate enzyme(s) at 345. Compound structural similarity may serve as a proxy for enzyme sharing. Therefore, the enzyme prediction module 330 may identify the sub-class and serial number of candidate enzyme(s) by running a molecular similarity search against substrates associated with the predicted subclass or EC number. The similarity between two substrates may be defined by the Tanimoto coefficient of two-dimensional (2D) fingerprints of the two compounds. Thus, at 350, the enzyme prediction module 330 finds similar compounds to the target drug in the enzyme-subclass category using Tanimoto coefficients.

The prediction filtering module 360 then performs molecular docking simulations at 365. For example, the prediction filter module 360 may identify binding sites and interaction orientations through molecular docking. In particular, the prediction filtering module 360 may obtain crystal structure(s) of the predicted enzyme(s) from an enzyme database, such as the BRENDA enzyme database which includes tens of thousands of three-dimensional enzyme structures across thousands of EC classes. The prediction filter module 360 may include the Glide docking program to perform accurate docking analysis of enzymes and substrates. The target drug compound 332 may be docked considering its flexibility by ligand conformational sampling method implemented in Glide, for example. The prediction filter module 360 may obtain multiple poses per enzyme-drug pair. The five best poses for a given enzyme-drug pair may be determined by a highest value of GlideScore, the empirical scoring function in Glide that approximates the ligand binding free energy.

At 370, the prediction filter module 360 performs molecular dynamics simulations and calculates free energy to identify the best enzyme candidates. Performing molecular dynamics simulations may identify false positive predictions of enzymes that may not have been filtered by the enzyme prediction module 330, and provide mechanistic insights about enzyme-drug binding by identifying key contacts between substrates and the enzyme catalytic pocket. The prediction filter module 360 may use GROMACS and CHARMM36 force field for molecular dynamics simulations, as illustrative and non-limiting examples. A regular molecular dynamics protocol may include solvation, neutralization of the net charge, minimization, and equilibration for one nanosecond using the NPT ensemble. The prediction filter module 360 may use a particle mesh Ewald method to calculate electrostatic interactions. The prediction filter module 360 may apply periodic boundary conditions in all three directions, use a time step of 2 femtoseconds, and maintain temperature at 310K and pressure at 1 bar. All molecular dynamics simulations may be run for one microsecond, for example, using the NPT ensemble.

The prediction filtering module 330 further identifies homologues of the identified enzyme(s) at 375, which will potentially have the same drug-metabolizing capacity. As an example, the homologues of the enzyme(s) could be identified using MetaPhOrs, a public repository of phylogeny-based orthologs and paralogs that were computed using phylogenetic trees available in twelve public repositories. Sequence-based homologue searches can be carried out using the cut-off values of Identity>80% and E-value<10⁻¹⁵ to identify microorganisms that encode homologues of the target enzyme. These cut-off values may be further calibrated.

Based on the molecular dynamics and free energy calculation at 370 and homology identification at 375, method 300 thus outputs predicted enzyme(s) responsible for metabolism of the target drug by the gut microbiome at 380. By integrating three-dimensional modeling methods and high-throughput fingerprint-based techniques as discussed hereinabove, accurate predictions of drug-metabolizing enzymes within the gut microbiome are obtained.

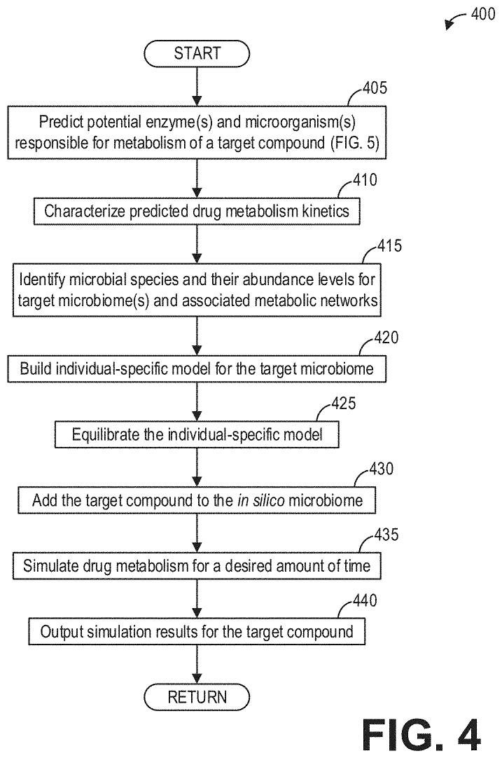

FIG. 4 shows a high-level flow chart illustrating an example method 400 for predicting gut microbiome-mediated drug metabolism, according to an embodiment. In particular, method 400 relates to leveraging deep learning models to predict potential enzymes responsible for metabolism of a target drug compound, integrating metagenomic and metatranscriptomic or metaproteomic data into a three-dimensional agent-based model for a human gut microbiome, and providing an in silico experimental platform to explore the interplay between a target drug compound and the gut microbiota. Method 400 is described with regard to the systems, components, and methods of FIGS. 1-3, though it should be appreciated that the method 400 may be implemented with other systems, components, and methods without departing from the scope of the present disclosure. Method 400 may be implemented as executable instructions in non-transitory memory of a data-holding subsystem 104 of a computing system 100, for example, and may be executed by a processor of a logic subsystem 102 of the computing system 100 to perform the actions described herein.

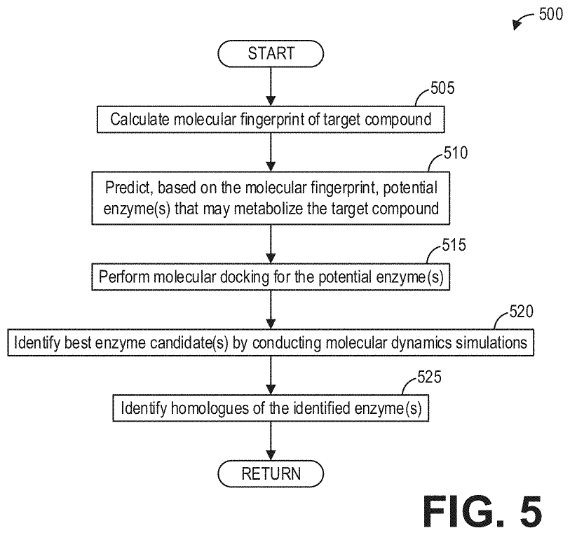

Method 400 begins at 405. At 405, method 400 predicts potential enzyme(s) and/or microorganism(s) responsible for metabolism of a target compound. As an illustrative and non-limiting example, FIG. 5 shows an example method 500 for predicting potential enzymes responsible for metabolism of a target compound. Method 500 begins at 505. At 505, method 500 calculates a molecular fingerprint for a target compound. At 510, method 500 predicts, with a trained deep learning model based on the molecular fingerprint, one or more potential enzyme(s) that may metabolize the target compound. At 515, method 500 performs molecular docking for the potential enzyme(s). At 520, method 500 identifies the best enzyme candidate(s) by conducting molecular dynamics simulations. At 525, method 500 identifies homologues of the identified enzyme(s). Method 500 then returns.

Referring again to FIG. 4, after predicting the potential enzymes responsible for metabolism of the target compound, method 400 continues to 410. At 410, method 400 characterizes predicted drug metabolism kinetics. For example, method 400 may import empirical degradation data for the target drug compound in order to validate the predicted metabolism. As discussed hereinabove, the empirical degradation data may be obtained via in vitro monoculture experiments.

At 415, method 400 identifies microbial species and their abundance levels for target microbiome(s) and associated metabolic networks. For example, method 400 may extract types and abundance levels of microbial species and metabolic networks from metagenomic data as inputs for the in silico experimental platform. To that end, method 400 may obtain raw metagenomics data through sequencing the target microbiome or through genome databases, map the sequenced reads to a set of reference genomes, and calculate the abundance of each species by adding up sequence length of reads mapped to a unique region of a species' genome, normalized by the total size of the species' genome. Method 400 may further use metabolic network datasets, in some examples, or in other examples method 400 may use the CarveMe tool, or other tools for metabolic network reconstruction, to build metabolic models using reference genomes. The reconstructed metabolic models may be associated with a corresponding agent type. Method 400 may further use flux balance analysis to calculate the flow of metabolites through biochemical reactions in a metabolic network, for example as discussed hereinabove. Using flux balance analysis, method 400 determines growth rate, and updates metabolite concentrations according to the consumed and secreted metabolites by the microbial species. In order to further improve the accuracy of the analysis, method 400 may employ metatranscriptomics or metaproteomics as a complementary approach to refine metabolic models reconstructed based on metagenomic data.

Continuing at 420, method 400 builds an individual-specific model for the target microbiome. At 425, method 400 equilibrates the individual-specific model for at least twenty-four hours. At 430, method 400 adds the target compound to the in silico microbiome. At 435, method 400 simulates drug metabolism in the in silico microbiome for a desired amount of time. For example, method 400 constructs an individual-specific model of the target gut microbiome in interaction with the target drug compound. As discussed hereinabove, inputs to the individual model may include simulation parameters such as the size of the system (e.g., in micrometers), the time step (e.g., in seconds), and the number of desired simulation steps. Additional inputs may include molecular fields in the system (e.g., the target drug compound), their diffusion coefficients, and their initial concentrations. Additional inputs to the individual model include microorganisms, including abundances, size, and associated metabolic networks. Method 400 then constructs a three-dimensional environment of the simulation where microorganisms are randomly distributed throughout the environment. The modeling environment may be discretized at the molecular scale and the initial concentration of molecular fields may be assigned to each grid cell. In addition to characteristics of agents, the agent-based model may provide other aspects of the simulation such as environmental boundaries, physical factors (e.g., crowding and steric repulsion), and collision detection. Molecular species may be modeled using ordinary differential equation (ODE)-based methods. For example, the three-dimensional environment may be discretized into uniform boxes and molecules, modeled using ODEs, and allowed to diffuse between boxes with the diffusion of molecules governed by Fick's Second Law. Diffusion may be modeled using the Grajdeanu algorithm, as an example. The concentration in each grid cell may depend on the concentration in neighboring grid cells, the distance between cells, and the diffusion coefficient. At each time step, molecular fields may be evaluated and field concentrations may be updated according to degradation via the present organisms.

Method 400 then runs the simulation for the desired number of time steps. At each time step, a range of data may be stored such as coordinates of microorganisms, cell population, and the concentration of molecular fields. Microorganisms may be represented by autonomous agents possessing cellular characteristics including growth, division, and migration. Microorganism growth, death, and division rules and rates may be naturally calculated from metabolic interactions or implemented based on experimental studies of morphogenesis in individual bacteria. At 440, method 400 outputs simulation results for the target compound. The simulation results may include the results acquired over the time steps. Method 400 then returns.

To demonstrate the accuracy and advantages of the systems and methods provided herein relative to previous approaches, the results of multiple modeling and analysis studies that are illustrated in FIGS. 7-9. First, fifteen different human gut microbiome samples were simulated in the absence of drugs to demonstrate the ability to accurately represent the gut microbiome. Second, drug concentration changes were predicted over time for in vitro metabolism of two cardiovascular drugs (digoxin and diltiazem). Third, diltiazem concentration changes over time were predicted for its ex vivo metabolism by three different human fecal samples over twenty-four hours.

For each simulated microbiome sample, it is expected that, in the absence of external stimuli, the composition of the microbiome over the time of simulation would not deviate from its initial composition. FIG. 7 depicts a set of graphs 700 illustrating results for twenty-four hour in silico experiments for fifteen human fecal samples, including 10 samples from a study of early-onset Crohn's disease (CD) patients and 5 samples from a cohort of individuals with allergic diseases who participated in a Phase I clinical trial. For the CD study, paired-end Illumina raw reads for five healthy controls and five CD patients were retrieved from NCBI SRA under the accession SRP057027. For allergic patients, paired-end Illumina raw reads were provided by Siolta Therapeutics, Inc. Raw reads underwent pre-processing and analysis using the microbiome characterization module 242. Each microbiome was constructed using the agent-based model(s) module 248 and simulated for 24 hours with a time step of one hour. Alpha diversity and Aitchison distance were monitored throughout the simulation. Alpha diversity was calculated using the Shannon diversity index, and is depicted in the graph 705. Aitchison distance was calculated by taking the Euclidean distance between the centered-log transformed samples, and is depicted in the graph 710. FIG. 7 depicts that all the microbiome samples show a change of <10% in Shannon index throughout the simulation and an Aitchison distance of <20 between the final and initial composition of the simulated microbiome, confirming that the complex, multiscale dynamics of the human gut microbiota is captured over time.

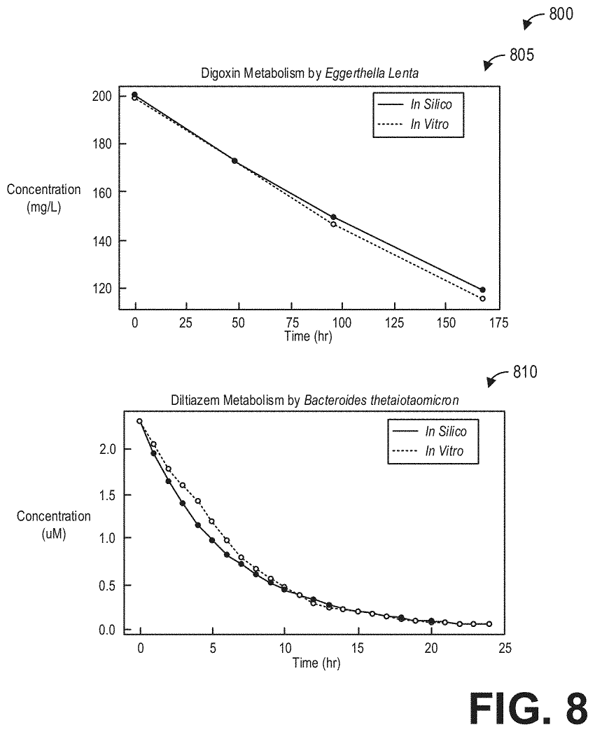

FIG. 8 shows a set of graphs 800 including two graphs 805 and 810 illustrating example drug metabolism kinetics measured during in silico experiments and in vitro monoculture experiments with Eggerthella lenta and Bacteroides thetaiotaomicron metabolizing digoxin and diltiazem, respectively. For in vitro metabolism of digoxin by Eggerthella lenta, a series of microcosm studies were conducted in serum bottle experiments under anaerobic conditions. E. lenta DSM 2243 was obtained as freeze-dried cells and was grown with Bacto Brain Heart Infusion broth amended with 1% arginine to enhance the growth rate. The bacteria were grown in a 125 mL sterile serum bottle with 50 mL of media inside an anaerobic chamber under a headspace of 5% H₂, 15% CO₂ and balance N₂. After 48 hours of growth, bacterial cells were used to perform degradation studies (with 200 mg/L of digoxin). Samples were collected every 48 hours until 168 hours and analyzed. Specifically, digoxin was analyzed by first extracting the compound from the filtered sample using chloroform as an extractant. Extraction was performed two times with a ratio 2:1 of chloroform to sample. After extraction, the chloroform layer was used for calorimetric analysis of digoxin. In 3 mL of chloroform extract, 3 mL of glacial acetic acid was added followed by 1.5 mL concentrated sulfuric acid dropwise on the side of the test tube. After 10 minutes of reaction, absorbance of the sample (measured using a Spectrophotometer at 490 nm) was used to calculate the concentration using a calibration curve prepared with digoxin standards. For in vitro metabolism of diltiazem by Bacteroides thetaiotaomicron reference data was obtained from a study on microbial metabolism of diltiazem. Twenty-four-hour time course data was obtained for in vitro metabolism by B. thetaiotaomicron. Metabolites associated with the metabolic network of each microorganism were added to the in silico experiment with an initial concentration of 0.5 mM. Simulations were run for 168 and twenty-four hours with a time step of one hour for digoxin and diltiazem experiments, respectively. The concentrations of drugs were compared to experimental values. The experimental metabolism trend over time for both drugs was predicted with an average error of 1.47% for digoxin and 2.24% for diltiazem.

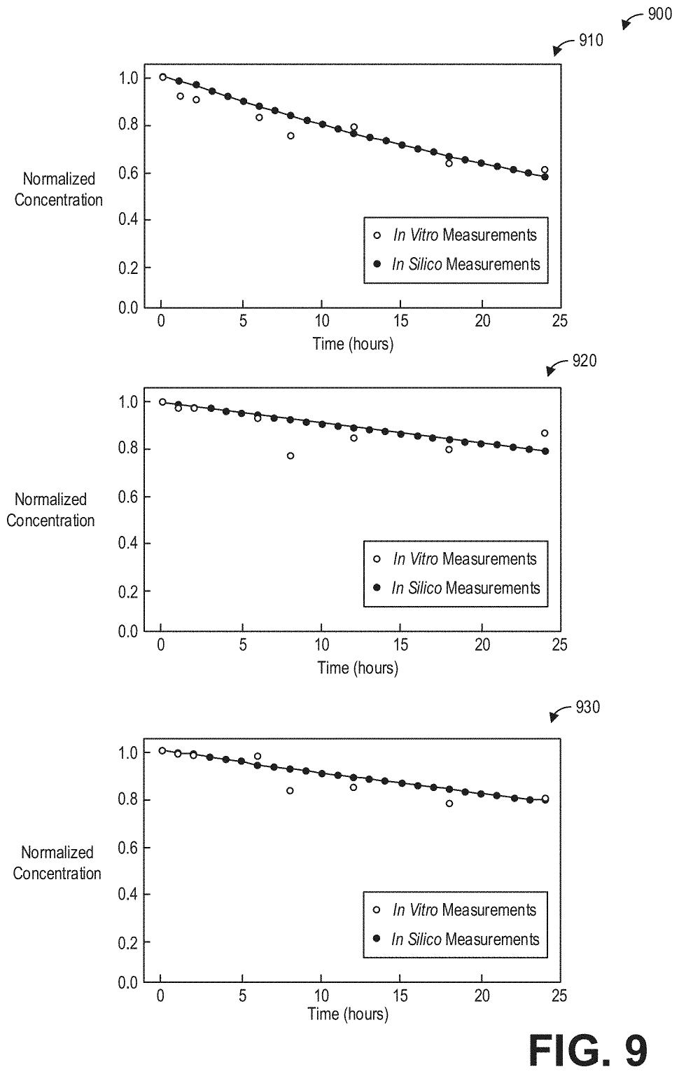

FIG. 9 shows a set of graphs 900 illustrating example drug metabolism kinetics measured during in silico experiments and in vitro experiments of diltiazem, according to an embodiment. The graphs depict results for the in vitro experiment as well as results for the in silico experiment, in addition to a legend distinguishing the markers for depicting the results. For both the in silico experiments and in vitro experiments, human microbiome samples from three different individual were used to characterize ex vivo metabolism of diltiazem. The graphs 910, 920, and 930 thus correspond to the samples from the three different individuals. Reference data for in vitro experiment was obtained from a recent study on microbial metabolism of diltiazem. Twenty-four-hour time course data was obtained for ex vivo metabolism by three fecal samples from healthy individuals that were collected using identifier numbers that are not associated with study volunteer names or other identifying information. Each sample was processed using the microbiome characterization module 232. An individual-specific model of each sample was built using the agent-based model(s) module 238. Diltiazem initial concentration was set to 2.3 uM. Diltiazem metabolism was assigned to Bacteroides thetaiotaomicron. Exchange metabolites associated with each metabolic network were added to the model with an initial concentration of 2.5 mM to support the growth of all microorganisms. Each in silico microbiome was simulated for twenty-four hours in the absence of the drug to reach a steady state, followed by twenty-four hours of simulation in the presence of diltiazem. The concentration of diltiazem was compared to experimental values. The results demonstrate that the systems and methods provided herein accurately capture the individual-specific gut microbiome-mediate drug metabolism with an average error of 5.34%, 5.15%, and 3.70% for the three samples, respectively.

Further, the in silico experiment provides time-course data with an increased sampling frequency, as depicted, relative to in vitro or in vivo experiment. For example, in practice for in vivo experiments, the common approach is to identify the response of the gut microbiome to the administration of drugs by analyzing metagenomic data obtained only before and after introduction of the drug, and so the sampling frequency is substantially limited. In contrast, as the in silico experiment comprises a predictive approach, the time-course data with increased sampling frequency provides more detailed information about the underlying dynamics governing the functional and compositional landscape of the gut microbiome.

Further still, unlike prior approaches to understanding drug metabolism, the systems and methods provided herein enable clinically-relevant predictions in response to perturbations to input parameters, such as the composition of the microbiota over time responsive to adjustments or perturbations to the concentration of drug compounds. As an additional advantage, where prior methods may study gut microbiota at the population level, the systems and methods provided herein incorporate individual heterogeneity. For example, by employing agent-based modeling and efficiently accounting for heterogeneity in characteristics of individuals (e.g., movement and metabolic phenotypes), the systems and methods provided herein account for the fact that individual microbes of the same species are heterogeneous individuals that behave according to the metabolites available in their immediate environment.

In one embodiment, a method comprises predicting a plurality of enzymes potentially associated with metabolism of a chemical compound, performing molecular docking and molecular dynamics simulations for each member of the plurality of enzymes potentially associated with metabolism of the chemical compound to identify one or more compound-metabolizing enzymes, characterizing metabolism kinetics of the chemical compound by the one or more compound-metabolizing enzymes, building a three-dimensional model of a microbiome using data comprising one or more of metagenomic data, metatranscriptomic data, and metaproteomic data, and simulating chemical compound metabolism by the three-dimensional model of the microbiome over time, wherein the three-dimensional model of the microbiome comprises a plurality of microorganisms including the microorganisms associated with the one or more compound-metabolizing enzymes.

In a first example of the method, the method further comprises predicting the plurality of enzymes with a trained artificial neural network. In a second example of the method optionally including the first example, the trained artificial neural network is a trained deep neural network. In a third example of the method optionally including one or more of the first and second examples, the plurality of enzymes potentially associated with metabolism of the chemical compound are each associated with a four-digit Enzyme Commission (EC) number. In a fourth example of the method optionally including one or more of the first through third examples, the method further comprises calculating a molecular fingerprint for the chemical compound, wherein the predicting is at least partly based on the molecular fingerprint. In a fifth example of the method optionally including one or more of the first through fourth examples, the microbiome comprises a gut microbiome. In a sixth example of the method optionally including one or more of the first through fifth examples, the chemical compound comprises a drug compound. In a seventh example of the method optionally including one or more of the first through sixth examples, the gut microbiome is individualized to a subject prescribed the drug compound. In an eighth example of the method optionally including one or more of the first through seventh examples, the simulating comprises updating, at each time step of a plurality of time steps, coordinates of the chemical compound metabolism, wherein the coordinates comprise a microorganism identity and a concentration of a molecular field corresponding to the chemical compound within the three-dimensional model. In a ninth example of the method optionally including one or more of the first through eighth examples, the method further comprises updating the concentration of the molecular field according to experimentally-characterized degradation kinetics for the chemical compound. In a tenth example of the method optionally including one or more of the first through ninth examples, the experimentally-characterized degradation kinetics for the chemical compound are measured based on an in vitro monoculture experiment. In an eleventh example of the method optionally including one or more of the first through tenth examples, the method further comprises determining, based on the simulating, degradation data for the chemical compound and one or more of metabolites of the chemical compound as a result of metabolism of the chemical compound by the microbiome. In a twelfth example of the method optionally including one or more of the first through eleventh examples, the method further comprises assigning a compound-metabolizing capacity to the microbiome based on the degradation data. In a thirteenth example of the method optionally including one or more of the first through twelfth examples, the method further comprises determining, based on the predicting and the performing, potential microbial metabolism of the chemical compound. In a fourteenth example of the method optionally including one or more of the first through thirteenth example, the method further comprises characterizing, based on the simulating, a change in microbiome composition as a result of interaction with the chemical compound.

In another embodiment, a method comprises predicting, with a trained deep neural network, a plurality of enzymes potentially associated with metabolism of a chemical compound, generating a three-dimensional individual-specific model of a microbiome, wherein the three-dimensional individual-specific model of the microbiome comprises a plurality of microorganisms including microorganisms associated with the plurality of enzymes, and simulating, with the three-dimensional individual-specific model of the microbiome, metabolism of the chemical compound in the microbiome over time.

In a first example of the method, the microbiome comprises a gut microbiome. In a second example of the method optionally including the first example, the chemical compound comprises a drug compound. In a third example of the method optionally including one or more of the first and second examples, the gut microbiome is individualized to a subject prescribed the drug compound. In a fourth example of the method optionally including one or more of the first through third examples, the method further comprises characterizing, based on the simulating, a change in microbiome composition as a result of interaction with the chemical compound. In a fifth example of the method optionally including one or more of the first through fourth examples, the plurality of enzymes are each associated with a four-digit Enzyme Commission (EC) number. In a sixth example of the method optionally including one or more of the first through fifth examples, the method further comprises calculating a molecular fingerprint for the chemical compound, wherein the predicting is at least partly based on the molecular fingerprint. In a seventh example of the method optionally including one or more of the first through sixth examples, the predicting comprises calculating a molecular fingerprint for the chemical compound, inputting the molecular fingerprint into the trained deep neural network, receiving, from the trained deep neural network, a prediction of one or more enzyme classes and one or more subclasses, running a molecular similarity search against enzyme substrates to identify sub-subclass and serial number of the plurality of enzymes, and identifying homologues of the plurality of enzymes that potentially have a same compound-metabolizing capacity. In an eighth example of the method optionally including one or more of the first through seventh examples, the predicting further comprises performing molecular docking and molecular dynamics to filter the plurality of enzymes to obtain a filtered candidate enzyme for metabolism of the chemical compound. In a ninth example of the method optionally including one or more of the first through eighth examples, the generating comprises identifying microorganisms and their relative abundances present in the target microbiome, obtaining or reconstructing metabolic networks for the identified microorganisms, and generating the three-dimensional individual-specific model of the microbiome including the identified microorganisms using agent-based modeling. In a tenth example of the method optionally including one or more of the first through ninth examples, the simulating comprises updating, at each time step of a plurality of time steps, coordinates of the plurality of microorganisms and concentrations of molecular fields corresponding to metabolites and the chemical compound within the three-dimensional individual-specific model, and performing, at each time step of a plurality of time steps, flux balance analysis for each microorganism to predict growth and replication of the microorganism. In an eleventh example of the method optionally including one or more of the first through tenth examples, the method further comprises updating the concentrations of the molecular fields according to experimentally-characterized degradation kinetics for the chemical compound measured based on in vitro monoculture experiments. In a twelfth example of the method optionally including one or more of the first through thirteenth examples, the method further comprises determining, based on the simulating, degradation data for the chemical compound and one or more metabolites of the chemical compound as a result of metabolism of the chemical compound by the microbiome. In a thirteenth example of the method optionally including one or more of the first through twelfth examples, the method further comprises assigning a compound-metabolizing capacity to the microbiome based on the degradation data.

In yet another embodiment, a system comprises a processor, and a non-transitory memory storing executable instructions that when executed cause the processor to: predict, with a trained deep neural network, a plurality of enzymes potentially associated with metabolism of a chemical compound; generate a three-dimensional individual-specific model of a microbiome including one or more microorganisms associated with the plurality of enzymes; and simulate, with the three-dimensional individual-specific model, metabolism of the chemical compound in the microbiome over time.

In a first example of the system, to predict the plurality of enzymes, the non-transitory memory further stores executable instructions that when executed cause the processor to: calculate a molecular fingerprint for the chemical compound; input the molecular fingerprint to the trained deep neural network; receive, from the trained deep neural network, a prediction of enzyme classes and subclasses; perform a molecular similarity search against substrates associated with the predicted enzyme subclasses to identify enzyme sub-subclasses and serial numbers; and perform molecular docking and molecular dynamics simulations to filter candidate enzymes to identify one or more compound-metabolizing enzymes. In a second example of the system optionally including the first example, to generate the three-dimensional individual-specific model of the microbiome including the one or more microorganisms associated with the plurality of enzymes, the non-transitory memory further stores executable instructions that when executed cause the processor to: construct metabolic models for the one or more microorganisms; perform flux balance analysis for each microorganism to predict growth and replication of the microorganism; and generate the three-dimensional individual-specific model of the microbiome using agent-based modeling. In a third example of the system optionally including one or more of the first and second examples, to simulate, with the three-dimensional individual-specific model, metabolism of the chemical compound by the microbiome over time, the non-transitory memory further stores executable instructions that when executed cause the processor to: update, at each time step of a plurality of time steps, coordinates of one or more microorganisms and concentrations of molecular fields corresponding to metabolites and the chemical compound within the three-dimensional individual-specific model. In a fourth example of the system optionally including one or more of the first through third examples, the non-transitory memory further stores executable instructions that when executed cause the processor to update the concentrations of the molecular fields according to experimentally-characterized degradation kinetics for the chemical compound measured based on in vitro monoculture experiments. In a fifth example of the system optionally including one or more of the first through fourth examples, the non-transitory memory further stores executable instructions that when executed cause the processor to: determine potential microbial metabolism of the chemical compound; determine degradation data for the chemical compound and one or more metabolites of the chemical compound as a result of metabolism of the chemical compound by the microbiome; assign a compound-metabolizing capacity to the microbiome based on the degradation data; and characterize a change in microbiome composition as a result of interaction with the chemical compound. In a sixth example of the system optionally including one or more of the first through fifth examples, the microbiome comprises a gut microbiome. In a seventh example of the system optionally including one or more of the first through sixth examples, the chemical compound comprises a drug compound.

Aspects of the disclosure may operate on particularly created hardware, firmware, digital signal processors, or on a specially programmed computer including a processor operating according to programmed instructions. The terms controller or processor as used herein are intended to include microprocessors, microcomputers, Application Specific Integrated Circuits (ASICs), and dedicated hardware controllers.

One or more aspects of the disclosure may be embodied in computer-usable data and computer-executable instructions, such as in one or more program modules, executed by one or more computers (including monitoring modules), or other devices. Generally, program modules include routines, programs, objects, components, data structures, and so on, that perform particular tasks or implement particular abstract data types when executed by a processor in a computer or other device. The computer executable instructions may be stored on a computer readable storage medium such as a hard disk, optical disk, removable storage media, solid state memory, Random Access Memory (RAM), etc. As will be appreciated by one of skill in the art, the functionality of the program modules may be combined or distributed as desired in various aspects. In addition, the functionality may be embodied in whole or in part in firmware or hardware equivalents such as integrated circuits, FPGAs, and the like.

Particular data structures may be used to more effectively implement one or more aspects of the disclosure, and such data structures are contemplated within the scope of computer executable instructions and computer-usable data described herein.

The disclosed aspects may be implemented, in some cases, in hardware, firmware, software, or any combination thereof. The disclosed aspects may also be implemented as instructions carried by or stored on one or more or computer-readable storage media, which may be read and executed by one or more processors. Such instructions may be referred to as a computer program product. Computer-readable media, as discussed herein, means any media that can be accessed by a computing device. By way of example, and not limitation, computer-readable media may comprise computer storage media and communication media.

Computer storage media means any medium that can be used to store computer-readable information. By way of example, and not limitation, computer storage media may include RAM, ROM, Electrically Erasable Programmable Read-Only Memory (EEPROM), flash memory or other memory technology, Compact Disc Read Only Memory (CD-ROM), Digital Video Disc (DVD), or other optical disk storage, magnetic cassettes, magnetic tape, magnetic disk storage or other magnetic storage devices, and any other volatile or nonvolatile, removable or non-removable media implemented in any technology. Computer storage media excludes signals per se and transitory forms of signal transmission.

Communication media means any media that can be used for the communication of computer-readable information. By way of example, and not limitation, communication media may include coaxial cables, fiber-optic cables, air, or any other media suitable for the communication of electrical, optical, Radio Frequency (RF), infrared, acoustic or other types of signals.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well of any dividual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well of any dividual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

The previously described versions of the disclosed subject matter have many advantages that were either described or would be apparent to a person of ordinary skill. Even so, these advantages or features are not required in all versions of the disclosed apparatus, systems, or methods.

Additionally, this written description makes reference to particular features. It is to be understood that the disclosure in this specification includes all possible combinations of those particular features. Where a particular feature is disclosed in the context of a particular aspect or example, that feature can also be used, to the extent possible, in the context of other aspects and examples.

Also, when reference is made in this application to a method having two or more defined steps or operations, the defined steps or operations can be carried out in any order or simultaneously, unless the context excludes those possibilities.

Although specific examples of the invention have been illustrated and described for purposes of illustration, it will be understood that various modifications may be made without departing from the spirit and scope of the invention.

1. A method, comprising:
predicting a plurality of enzymes potentially associated with metabolism of a chemical compound;
performing molecular docking and molecular dynamics simulations for each member of the plurality of enzymes potentially associated with metabolism of the chemical compound to identify one or more compound-metabolizing enzymes;
characterizing metabolism kinetics of the chemical compound by the one or more compound-metabolizing enzymes;
building a three-dimensional model of a microbiome using data comprising one or more of metagenomic data, metatranscriptomic data, and metaproteomic data; and
simulating chemical compound metabolism by the three-dimensional model of the microbiome over time, wherein the three-dimensional model of the microbiome comprises a plurality of microorganisms including the microorganisms associated with the one or more compound-metabolizing enzymes.

2. The method of claim 1, further comprising predicting the plurality of enzymes with a trained artificial neural network.

3. The method of claim 2, wherein the trained artificial neural network is a trained deep neural network.

4. The method of claim 3, wherein the trained deep neural network predicts a four-digit Enzyme Commission (EC) number for each enzyme of the plurality of enzymes potentially associated with metabolism of the chemical compound.

5. The method of claim 1, further comprising calculating a molecular fingerprint for the chemical compound, wherein the predicting is at least partly based on the molecular fingerprint.

6. The method of claim 1, wherein the microbiome comprises a gut microbiome, the chemical compound comprises a drug compound, and the gut microbiome is individualized to a subject prescribed the drug compound.

7. The method of claim 1, wherein the simulating comprises:
updating, at each time step of a plurality of time steps, coordinates of the chemical compound metabolism, wherein the coordinates comprise a microorganism identity and a concentration of a molecular field corresponding to the chemical compound within the three-dimensional model.

8. The method of claim 7, further comprising updating the concentration of the molecular field according to experimentally-characterized degradation kinetics for the chemical compound.

9. The method of claim 8, wherein the experimentally-characterized degradation kinetics for the chemical compound are measured based on an in vitro monoculture experiment.

10. The method of claim 9, further comprising:
determining, based on the simulating, degradation data for the chemical compound and one or more of metabolites of the chemical compound as a result of metabolism of the chemical compound by the microbiome.

11. The method of claim 10, further comprising:
assigning a compound-metabolizing capacity to the microbiome based on the degradation data.

12. The method of claim 1, further comprising:
determining, based on the predicting and the performing, potential microbial metabolism of the chemical compound.

13. The method of claim 1, further comprising:
characterizing, based on the simulating, a change in microbiome composition as a result of interaction with the chemical compound.

14. A method, comprising:
predicting, with a trained deep neural network, a plurality of enzymes potentially associated with metabolism of a chemical compound;
generating a three-dimensional individual-specific model of a microbiome, wherein the three-dimensional individual-specific model of the microbiome comprises a plurality of microorganisms including microorganisms associated with the plurality of enzymes; and
simulating, with the three-dimensional individual-specific model of the microbiome, metabolism of the chemical compound in the microbiome over time.

15. The method of claim 14, wherein the microbiome comprises a gut microbiome, the chemical compound comprises a drug compound, and the gut microbiome is individualized to a subject prescribed the drug compound.

16. The method of claim 14, further comprising:
characterizing, based on the simulating, a change in microbiome composition as a result of interaction with the chemical compound.

17. The method of claim 14, wherein the trained deep neural network predicts a four-digit Enzyme Commission (EC) number for each enzyme of the plurality of enzymes potentially associated with metabolism of the chemical compound.

18. The method of claim 14, wherein the predicting comprises:
calculating a molecular fingerprint for the chemical compound;
inputting the molecular fingerprint into the trained deep neural network;
receiving, from the trained deep neural network, a prediction of one or more enzyme classes and one or more subclasses;
running a molecular similarity search against enzyme substrates to identify sub-subclass and serial number of the plurality of enzymes; and
identifying homologues of the plurality of enzymes that potentially have a same compound-metabolizing capacity.

19. The method of claim 18, wherein the predicting further comprises:
performing molecular docking and molecular dynamics to filter the plurality of enzymes to obtain a filtered candidate enzyme for metabolism of the chemical compound.

20. The method of claim 14, wherein the generating comprises:
identifying microorganisms and their relative abundances present in the microbiome;
obtaining or reconstructing metabolic networks for the identified microorganisms; and
generating the three-dimensional individual-specific model of the microbiome including the identified microorganisms using agent-based modeling.

21. The method of claim 14, wherein the simulating comprises:
updating, at each time step of a plurality of time steps, coordinates of the plurality of microorganisms and concentrations of molecular fields corresponding to metabolites and the chemical compound within the three-dimensional individual-specific model; and
performing, at each time step of a plurality of time steps, flux balance analysis for each microorganism to predict growth and replication of the microorganism.

22. The method of claim 21, further comprising updating the concentrations of the molecular fields according to experimentally-characterized degradation kinetics for the chemical compound measured based on in vitro monoculture experiments.

23. The method of claim 22, further comprising:
determining, based on the simulating, degradation data for the chemical compound and one or more metabolites of the chemical compound as a result of metabolism of the chemical compound by the microbiome.

24. The method of claim 23, further comprising:
assigning a compound-metabolizing capacity to the microbiome based on the degradation data.

25. A system comprising:
a processor; and
a non-transitory memory storing executable instructions that when executed cause the processor to:
predict, with a trained deep neural network, a plurality of enzymes potentially associated with metabolism of a chemical compound;
generate a three-dimensional individual-specific model of a microbiome including one or more microorganisms associated with the plurality of enzymes; and
simulate, with the three-dimensional individual-specific model, metabolism of the chemical compound in the microbiome over time.

26. The system of claim 25, wherein, to predict the plurality of enzymes, the non-transitory memory further stores executable instructions that when executed cause the processor to:
calculate a molecular fingerprint for the chemical compound;
input the molecular fingerprint to the trained deep neural network;
receive, from the trained deep neural network, a prediction of enzyme classes and subclasses;
perform a molecular similarity search against substrates associated with the predicted enzyme subclasses to identify enzyme sub-subclasses and serial numbers; and
perform molecular docking and molecular dynamics simulations to filter candidate enzymes to identify one or more compound-metabolizing enzymes.

27. The system of claim 25, wherein, to generate the three-dimensional individual-specific model of the microbiome including the one or more microorganisms associated with the plurality of enzymes, the non-transitory memory further stores executable instructions that when executed cause the processor to:
construct metabolic models for the one or more microorganisms;
perform flux balance analysis for each microorganism to predict growth and replication of the microorganism; and
generate the three-dimensional individual-specific model of the microbiome using agent-based modeling.

28. The system of claim 25, wherein, to simulate, with the three-dimensional individual-specific model, metabolism of the chemical compound by the microbiome over time, the non-transitory memory further stores executable instructions that when executed cause the processor to:
update, at each time step of a plurality of time steps, coordinates of one or more microorganisms and concentrations of molecular fields corresponding to metabolites and the chemical compound within the three-dimensional individual-specific model.

29. The system of claim 25, wherein the non-transitory memory further stores executable instructions that when executed cause the processor to:
update the concentrations of the molecular fields according to experimentally-characterized degradation kinetics for the chemical compound measured based on in vitro monoculture experiments.

30. The system of claim 25, wherein the non-transitory memory further stores executable instructions that when executed cause the processor to:
determine potential microbial metabolism of the chemical compound;
determine degradation data for the chemical compound and one or more metabolites of the chemical compound as a result of metabolism of the chemical compound by the microbiome;
assign a compound-metabolizing capacity to the microbiome based on the degradation data; and
characterize a change in microbiome composition as a result of interaction with the chemical compound.
